(19) United States
(12) Plant Patent Application Publication
(10) Pub. No.: US 2004/0154064 P1
(43) Pub. Date: Aug. 5, 2004

(54) Euphorbia milii plant named 'Zeus'

(75) Inventor: Steen Thomsen, Sonderso (DK)

Correspondence Address: FOLEY AND LARDNER, SUITE 500, 3000 K STREET NW, WASHINGTON, DC, 20007, US

(73) Assignee: Gartneriet Hjortebjerg I/S

(21) Appl. No.: 10/762,456

(22) Filed: Jan. 23, 2004

(30) Foreign Application Priority Data
Jan. 24, 2003 (EP) 2003/0125

(51) Int. Cl. A01H 5/00

(52) U.S. Cl. PLT/302

(57) ABSTRACT

A new distinct cultivar of Euphorbia milii plant named ‘Zeus’, particularly characterized by its compact plant habit; very low and bushy plant form; vigorous growth habit; brown streaked stems; abundant, large floral cymes with red bracts from 45C to bright deep red 50A; and more flowers on thick, stiff gray-brown peduncles N199D

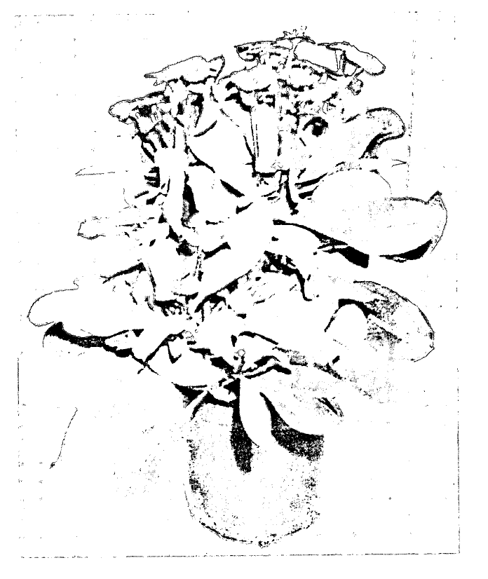

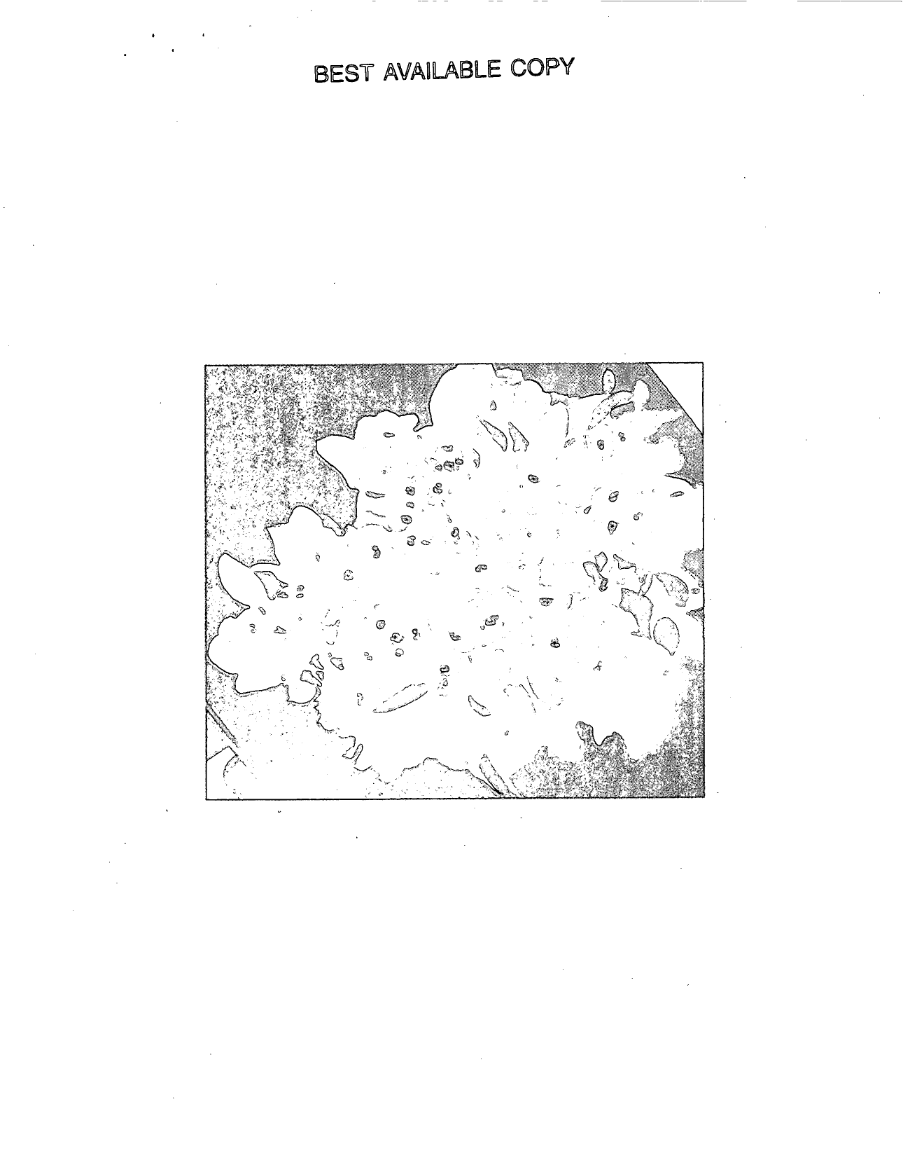

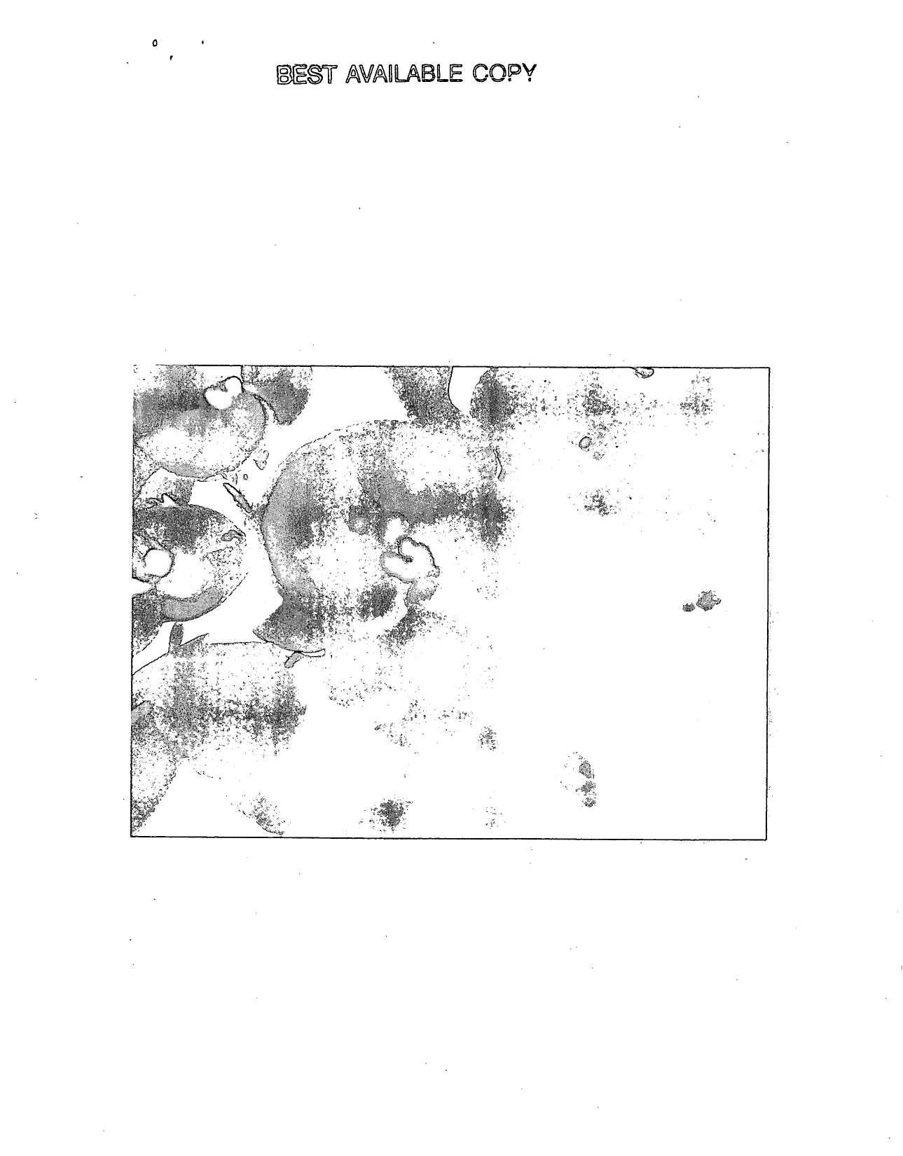

Genus and species of the plant claimed:

Euphorbia milii Desmoul.

Variety denomination:

Zeus

### BACKGROUND OF THE INVENTION

The present Invention relates to a new and distinct cultivar of Euphorbia milii plant, botanically known as Euphorbia milii Desmoul., commonly known as Crown of Thorns and hereinafter referred to by the name ‘Zeus’.

The new Euphorbia milii is a product of a planned breeding program conducted in Haarslev, Fyn, Denmark. The new Euphorbia milii originated from a polycross made in 2001 by the Inventor, Steen Thomsen, with cultivars of Euphorbia milii Desmoul. The parental cultivars are unnamed, unpatented cultivars of Euphorbia milii Desmoul. The Inventor selected the new Euphorbia milii cultivar from the progeny of the above crossing in 2001 on the basis of its flower color and compact, freely branching habit. Plants of the new Euphorbia milii are smaller, compact and have a unique color, medium sized leaves and abundant large flowers.

Asexual reproduction of the new cultivar by terminal cuttings taken and propagated and trial production batches at Hjortebjerg Nurseries, Denmark, has shown that the unique features of this new Euphorbia milii are stable and reproduced true to type in many successive generations of asexual reproduction.

### BRIEF DESCRIPTION OF THE INVENTION

The following traits have been repeatedly observed and are determined to be the unique characteristics of ‘Zeus’. These characteristics in combination distinguish ‘Zeus’ as a new and distinct cultivar:

1. Red floral bracts from 45C to bright deep red 50A.

2. Very low and bushy plant form.

3. Vigorous growth habit, brown streaked stems

4. More flowers on thick, stiff gray-brown peduncles—N199D

Plants of the cultivar ‘Zeus’ can be compared to plants of the cultivar Euphorbia milii. ‘Themis’. In side-by-side comparisons conducted by the Inventor in Haarslev, Denmark, plants of the cultivar ‘Zeus’ and the cultivar ‘Themis’ differed in the following characteristics:

1. Plants of the new Euphorbia milii have striking red colored bracts and larger cyathia with yellow glands, while ‘Themis’ has orange glands.

2. Plants of the new Euphorbia milii have shorter and stiffer peduncles than plants of the cultivar ‘Themis’.

3. Plants of the new Euphorbia milii are more compact and less branched than the plants of the cultivar ‘Themis’

4. Plants of the new Euphorbia milii have stiff, brown peduncles, while these are yellow-green on ‘Themis’

Plants of the cultivar ‘Zeus’ have not been observed under all possible environmental conditions. The phenotype may vary somewhat with variations in environment such as temperature, light intensity, day length, and fertility level without, however, any variance in genotype.

### BRIEF DESCRIPTION OF THE PHOTOGRAPHS

The accompanying colored photographs illustrate the overall appearance and details of flower form, color and structures of the new cultivar, showing the colors as true as it is reasonably possible to obtain in colored reproductions of this type. Colors in the photographs may differ slightly from the color values cited in the detailed botanical description, which more accurately describe the actual colors of the new Euphorbia milii.

The first photograph shows a side perspective view of a typical flowering plant of ‘Zeus’, as grown in 11 cm pots. The second photograph is a top view of flowers and foliage of ‘Zeus’. The third photograph is a close-up of the young and older floral cymes.

### DETAILED BOTANICAL DESCRIPTION

In the following description, color references are made to The Royal Horticultural Society Colour Chart, 4^{th} edition, where general terms of ordinary dictionary significance are used. Plants were grown under greenhouse conditions in Haarslev, Denmark. Plants used for this description were grown for about 17 weeks after cutting and produced in 6 cm pots. Other pot sizes can be used and the plants are intended for indoor use or as a bedding plant in temperate climates while it is a perennial garden plant in tropical and subtropical areas.

Botanical classification: Euphorbia milii Desmoul. cultivar ‘Zeus’. Euphorbiaceae, Spurge family. Common English name: Crown of Thorns.

Parentage:

Female parent.—Unnamed seedling plant of Euphorbia milii.

Male parent.—Unnamed seedling plant of Euphorbia milii.

Propagation:

Type cutting.—Terminal vegetative cuttings taken from plants kept in the vegetative stage by shading and high temperatures (25 C.).

Time to initiate roots.—About 10 to 14 days at 18 to 21 C. in tunnels in a greenhouse.

Root description.—Fine, well branched.

Plant description:

Form.—Perennial plant with upright plant habit. Euphorbia milii flowers in cymes with cyathia subtended by colored bracts. Freely branching with about 3 lateral flowering branches forming at every node; dense and bushy. Stems are square to pentagonal with ridges — about 10 mm thick at basis. By each node appear triple thorns: 1 large 10 mm and 1 smaller 4 mm. Young thorns are brown: 151A; while the older ones become stiffer and changes color to gray (197B).

Crop time.—After rooting, about 17 weeks are required to produce finished flowering plants in 6 cm pots.

Plant size (soil level to top of plant plane).—About 16 cm. Width: 17 cm.

Vigor.—Vigorous growth rate.

Foliage description: — Leaves alternate, single, obovate, entire, craspedromous venation. Length: 6 cm. Width: About 35 mm. Apex: acute. Base: cuneate to almost decussate Texture: smooth, waxy, dull, glabrous. Color: Young foliage, upper and lower surfaces: 139C and 145C, yellow-green. Mature foliage, upper and lower surfaces: 139A and 148C respectively. Venation, 148A.

Flower description:

Flower arrangement and shape.—Floral arrangements composed of cymes. The flowers (cyathia) are starkly reduced so only a circle of 5 glands and the reproductive organs are present. Subtending the cyathia are two colored bracts. The flowers are further complicated by the unique feature of funnel shaped floral buds appearing at the base of the bracts in two or more layers.

Natural flowering season.—Continuous throughout the spring and summer in subtropical and tropical regions. In colder climates season can be extended by greenhouse production with high temperatures and supplementary irradiance.

Flower longevity on the plant.—5 to 9 weeks; longevity of individual flowers is highly dependent on temperature and light conditions. Bracts turn green with age. Entire cymes drop after withering.

Inflorescence size.—Diameter: About 3×5 cm, height: 7 cm.

Flowers.—5 mm diameter, Bracts: overlapping at base, ovoid to inverted cordate 14×18 mm color 45C, red.

Glands.—5 from 5A shiny yellow to N30A, orange during development.

Pistil and stigma.—Appear before cyathia mature; color 45B, red.

Peduncle.—Strength: strong. Length: About 5 cm. Diameter: About 3 mm. Color: N199D, gray-brown at an angle of 40-70°.

Pedicels.—15 mm long, 2 mm thick strong color: N199C, brown.

Weather tolerance: Plants of the new Euphorbia milii have exhibited good tolerance to draught, rain and wind, however flowering may cease during cold and dark periods (<15 C.).

Pest tolerance: Plants of the new Euphorbia milii have exhibited good tolerance to following fungi: Mildew, and Thivaliopsis. Also they appear to be less infected by Thrips (Franklinellea).

I claim:

1. A new and distinct cultivar of Euphorbia milii plant named ‘Zeus’, as illustrated and described herein.
